(19) United States
(12) Patent Application Publication
(10) Pub. No.: US 2012/0283627 A1
(43) Pub. Date: Nov. 8, 2012

(54) METHOD FOR COMBINED GASTROINTESTIONAL FEEDING AND ASPIRATION

(76) Inventor: Gerald Moss, White Plains, NY (US)

(21) Appl. No.: 13/102,934

(22) Filed: May 6, 2011

(51) Int. Cl. A61M 1/00; A61M 31/00

(52) U.S. Cl. 604/28

(57) ABSTRACT

A method is provided in which continuous suction alternately removes fluid through an aspiration lumen from the stomach and adjacent small intestine and returns the fluid with gravity a short distance downstream. The only aspirate permanently discarded is that volume of inflow to the feeding site that momentarily exceeded peristaltic outflow. Feeding is also continuous, which allows for a smaller and more comfortable feeding tube. This frequent aspiration and feeding serves to provide the maximum safe nutrition, while preventing overfeeding and its associated risks of intestinal distention.

### BACKGROUND OF THE INVENTION

1. Field of the Invention

This invention relates to a method for continuously aspirating a patient's gastrointestinal tract of all swallowed air and excess inflow that arrives at the feeding site (feedings plus digestive juices), while safely delivering nutrients, fluids, medication and/or returned aspirate into a patient's gastrointestinal tract. More specifically, the invention relates to a method for continuously aspirating a patient's gastrointestinal tract and delivering nutrients, fluids, medication and/or aspirate into a patient's gastrointestinal tract via a combination feeding and aspiration tube.

2. Description of the Related Art

Nutrients, fluids, medications and/or returned aspirate (collectively referred to as “feedings”) often are introduced directly into a patient's proximal small intestine via an enteral feeding tube.

In many cases, a patient's digestive system is not operating fully, limiting the amount of nutrition the patient may receive. However, adequate nutrition is obviously a critical part of anyone's health, and is necessary to provide optimum recovery for a patient. It is thus desirable to deliver as much nutrition to a patient as can be absorbed safely by the patient's impaired gastrointestinal tract.

Unfortunately, it is not desirable simply to deliver a maximum amount of nutrition into a patient's intestine. The reason for this is that feeding at a rate which exceeds the ability of the patient's gastrointestinal tract to propel and absorb its own secretions plus added nutrition itself presents serious hazards to a patient. Overfeeding a patient leads to an accumulation of fluid, which distends the intestine at the feeding site. This local intestinal distension reflexly can lead to fatal circulatory changes. The more common complication from overfeeding is further impairment of gastrointestinal function, with generalized distention, nausea and vomiting, which at a minimum causes discomfort and delays recovery. Accordingly, it is desirable to limit delivery to avoid overfeeding, while at the same time delivering into the impaired intestine the maximum nutrition that can be absorbed safely.

Further complicating the nourishing of patients via an intestinal feeding tube is the fact that the body of its own accord produces a considerable volume of digestive secretions on a regular basis, approximately 7 to 8 liters/day. In a healthy person, all of these digestive secretions, starting with saliva, are re-absorbed by the intestine, leading to no net change in bodily fluids. These secretions contain antibodies specific against the patient's own enteric organisms, and provide protection against infection by them. During a patient's recovery from surgery or illness, the creation of digestive secretions is typically undiminished. Unfortunately, a patient's gastrointestinal function may be impaired considerably during recovery, so that the re-absorption of digestive secretions may not fully occur. Thus, nutrition delivered to the intestine may be competing with digestive secretions for absorption by the intestine. Furthermore, the presence of a feeding tube induces swallowing by a patient, which introduces additional air into the gastrointestinal tract. Swallowed air further compromises propulsion and absorption. Meanwhile, it is not desirable to simply remove digestive secretions from a patient's system, as this will dehydrate the patient. For this reason, feedings should be delivered to the patient in a way that accounts for any possible excess of digestive secretions, without dehydrating the patient or increasing his risk of infection by his own enteric bacteria, and while also minimizing the presence of air.

Previously, a feeding tube might be aspirated manually to “check for residual” as a safety measure. In this process, feeding is interrupted, and that portion of the patient's gastrointestinal tract is manually aspirated via the feeding tube. This “residual” volume is measured and compared with an expected volume for an individual with normal gastrointestinal function to determine whether the feeding rate should be maintained, increased, or reduced. The aspirate is either re-introduced or discarded. However, the check for residual process is labor intensive and, therefore, seldom used. Also, the check for residual process can, as a practical matter, be performed only a few times per day, which does not monitor gastrointestinal function as closely as desired when directly feeding into the intestine.

U.S. Pat. No. 6,447,472 describes a device for alternating the delivery of nutrition to a patient's gastrointestinal tract and aspirating air and excess fluid from the gastrointestinal tract using a positive displacement pump type assembly.

It has now been found that removing fluid and gas with suction and returning the fluid with gravity has advantages over using a positive displacement pump. First, a pump need not be provided for aspiration. A central vacuum pump is available in the hospital setting, and suction usually is available at the bedside. In addition, the maximum levels of suction and positive pressure produced by a positive displacement pump may be damaging to the patient and are inherently difficult to control.

### BRIEF SUMMARY OF THE INVENTION

The present invention is directed to a method for uninterrupted aspiration of the stomach and adjacent small intestine, with simultaneous delivery of nutrition, fluids, medicine and returned aspirate downstream into the slightly more distal small intestine. A combination feeding and aspiration tube is placed within a patient's gastrointestinal tract; wherein the patient is fed using the combination feeding and aspiration tube within the gastrointestinal tract. Liquid nutrition independently is delivered continuously by gravity or a pump. The stomach and adjacent small intestine are aspirated continuously using the combination feeding and aspiration tube, wherein the aspirate alternately flows into one of two reservoirs, wherein a first reservoir is initially open to room air and a second reservoir is initially on suction, under vacuum, wherein the reservoir on suction aspirates from a stomach and the adjacent small intestine. Simultaneously, the reservoir open to the room air (off suction) delivers its previously collected degassed liquid content by gravity via the feeding lumen to a point slightly beyond the area of aspiration but still within the same segment of the aspirated intestine.

The invention therefore provides a method for aspiration of a gastrointestinal tract and delivery of nutrition, fluids, medicine and aspirate to the gastrointestinal tract, the method comprising the steps of: placing a combination feeding and aspiration tube within a patient's gastrointestinal tract; feeding using the combination feeding and aspiration tube within the gastrointestinal tract; and aspirating the gastrointestinal tract using the combination feeding and aspiration tube, wherein the aspirate alternately flows into one of two reservoirs, wherein a first reservoir is open to room air and a second reservoir is on suction, wherein the second reservoir on suction aspirates from a stomach and adjacent small intestine, while the first reservoir open to the room air simultaneously delivers its degassed liquid content by gravity, which had been collected during the previous half-cycle, to a point downstream of aspiration area but within a same segment of the adjacent small intestine.

These and other features, aspects, and advantages of the present invention will become better understood with reference to the following drawings and description.

### BRIEF DESCRIPTION OF THE DRAWINGS

FIG. 1 is a diagrammatic view of a system in accordance with the present invention depicting feeding and aspirating in a gastrointestinal tract of a patient.

### DETAILED DESCRIPTION OF THE INVENTION

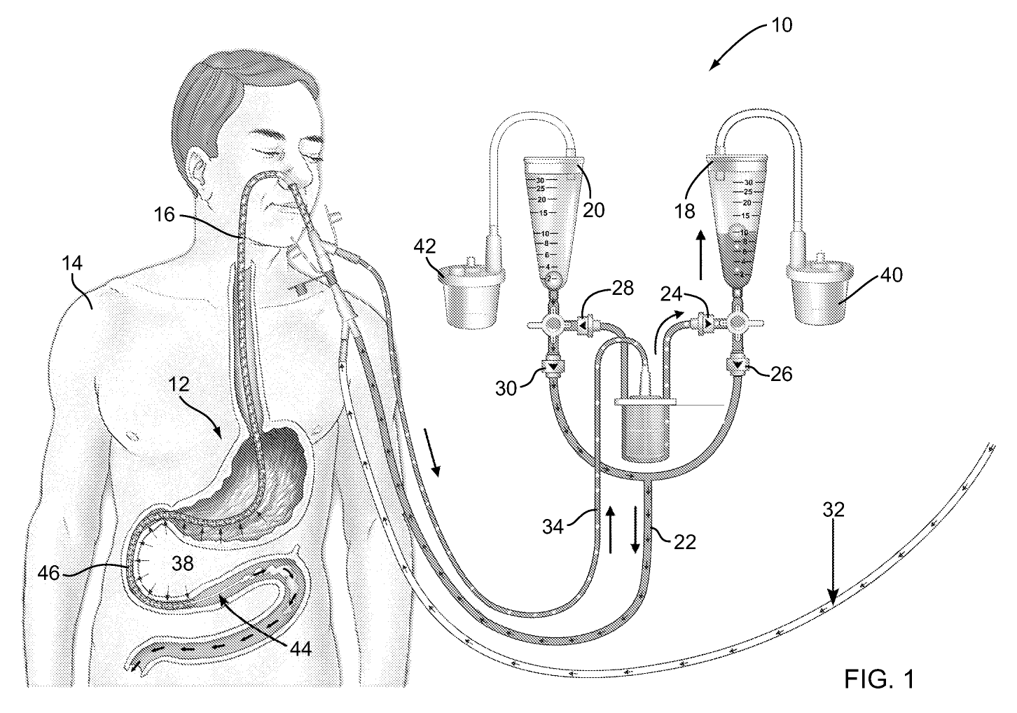

The present invention relates to a method for safely feeding and aspirating in a gastrointestinal tract. As shown in FIG. 1, an automatic feeding monitor/manager device 10 is used to feed and aspirate in a gastrointestinal tract 12 of a patient 14. To this end a combination feeding and aspirating tube 16 may be inserted into the gastrointestinal tract 12.

Two separate tubes, one for aspiration and one for delivery of feedings or a double lumen tube, wherein one lumen provides feedings and a second lumen provides the aspiration function may be used with the device of the present invention. The dual tubes may be side by side or coaxial in nature.

A flasher control (not shown) splits a cycle period, for example about 60 to about 300 seconds, in half for activation and de-activation of a four port solenoid valve (not shown). This valve switches vacuum to one reservoir 18 while simultaneously connecting the other reservoir 20 to room air. After a half cycle period, for example about 30 to about 150 seconds, the flasher reverses those connections by the solenoid valve. Suction continues to aspirate the stomach and adjacent small intestine for about 30 to about 150 seconds, directing the aspirate into reservoir 20, while the opposite reservoir 18 delivers its degassed liquid content to the feeding tube by gravity via tubing 22. The direction of flow is controlled passively by two pairs of one way valves 24 and 26 and 28 and 30.

The liquid nutrition (elemental diet) is delivered continuously by gravity or a pump (not shown) via tubing 32.

The aspirate via tubing 34 is filtered in filter 36 to remove solids and mucus from the aspirate, and alternately flows into the first reservoir 18 or second reservoir 20, appropriately directed passively by one-way valves 24 and 28.

Continuous suction is applied to the stomach and adjacent small intestine, removing air and liquid content through the porous aspiration channel through aspiration orifices 38, into one or the other aspiration reservoirs 18 or 20, whichever is on suction during that halfcycle. Any volume of liquid inflow to the feeding site (nutrition plus digestive juices) that exceeds peristaltic outflow during any half cycle from that site overflows from the reservoir into its overflow chamber 40 and 42, to be permanently removed. The reservoir off suction returns its liquid content via tubing 22, connected to the feeding lumen 44, which is positioned slightly downstream of the aspiration lumen 46, but within the same segment of intestine, for example, whereby the surgeon positions the feeding lumen 44 and the aspiration lumen 46 in the same segment of the intestine as a step in a surgical procedure.

The method of the present invention, with simultaneous inflow and aspiration, has many advantages over alternating cycles of aspiration and feeding. Interrupting the flow requires that the same volumes must be accommodated during only half of the cycle, rather than the entire cycle. Using the device of the present invention, the size of the patient's feeding-decompression tube(s) can be reduced, and/or its flow rate increased, without the danger of increased delivery pressure.

One of the advantages of removing fluid and gas with hospital suction and returning the fluid with gravity, as disclosed in the present invention, is a separate suction pump need not be provided. Suction is available in a hospital setting. The maximum levels of suction and positive pressure used by, for example, a positive displacement pump are potentially damaging to the patient and inherently difficult to control.

Other advantages of the two chamber fluidic system of the present invention include, but are not limited to, is that excess aspirated fluid is removed only if it exceeds the rate of peristaltic outflow from the feeding site rather than if it exceeds an arbitrary set maximum aspiration volume, such as, for example, 30 ml., so that less aspirate is needlessly discarded or an excess may still be present. The aspiration of a patient to remove swallowed air and excess fluid inflow is continuous, rather than being interrupted for one half of the cycle. Swallowed air and excess fluid do not have the opportunity to be propelled downstream, beyond the reach of the suction, during the off phase. The aspirated segment of the stomach and adjacent proximal small intestine is continuously rather than intermittently emptied, so that it will more rapidly recover normal function. Further, feeding is continuous, rather than being interrupted for one half of the cycle, so that a smaller, more comfortable feeding tube can be utilized, and/or lower levels of potentially damaging suction and pressure can be utilized.

The embodiments of the invention described herein are exemplary and numerous modifications, variations and rearrangements can be readily envisioned to achieve substantially equivalent results, all of which are intended to be embraced within the spirit and scope of the invention.

1. A method for aspiration of a gastrointestinal tract and delivery of nutrition, fluids, medicine and aspirate to the gastrointestinal tract, the method comprising the steps of:
placing a combination feeding and aspiration tube within a patient's gastrointestinal tract;
feeding using the combination feeding and aspiration tube within the gastrointestinal tract; and
aspirating the gastrointestinal tract using the combination feeding and aspiration tube, wherein the aspirate alternately flows into one of two reservoirs, wherein a first reservoir is open to room air and a second reservoir is on suction, wherein the second reservoir on suction aspirates from a stomach and adjacent small intestine, while the first reservoir open to the room air simultaneously delivers its degassed liquid content by gravity to a point downstream of aspiration area but within a same segment of the adjacent small intestine.

2. The method of claim 1, wherein the aspiration is continuous.

3. The method of claim 1, wherein the aspirated gastrointestinal segment of the stomach and adjacent small intestine is continuously empty.

4. The method of claim 1, wherein the feeding is continuous.

5. The method of claim 1, wherein a portion of aspirated fluid is removed permanently only to the extent that it exceeds rate of peristaltic outflow from feeding site.

6. The method of claim 1, wherein the feeding is delivered by gravity.

7. The method of claim 1, wherein the feeding is delivered by pump.

8. The method of claim 1, wherein a feeding lumen is positioned downstream of an aspiration lumen within the same segment of the small intestine.
